(19) United States
(12) Patent Application Publication
(10) Pub. No.: US 2015/0265770 A1
(43) Pub. Date: Sep. 24, 2015

(54) MICROJET DRUG DELIVERY SYSTEM USING ERBIUM YAG LASER

(71) Applicant: SNU R&DB FOUNDATION, Seoul (KR)

(72) Inventor: Jai-Ick Yoh, Seoul (KR)

(21) Appl. No.: 14/420,387

(22) PCT Filed: Aug. 12, 2013
(86) PCT No.: PCT/KR2013/007248

§ 371 Date: Jun. 8, 2015

(30) Foreign Application Priority Data
Aug. 10, 2012 (KR) 10-2012-0087843

(51) Int. Cl. A61M 5/30; A61M 5/31; A61M 5/20

(52) U.S. Cl. CPC A61M 5/30; A61M 5/2053; A61M 5/31

(57) ABSTRACT

A microjet drug delivery system includes a pressure chamber which has a predetermined storing space and of which a closed inner portion is hermetically filled with water, a drug chamber which is disposed adjacent to the pressure chamber and is configured to store a drug solution in a predetermined storing space and where a micro nozzle ejecting the drug solution as a microjet to an outside is formed at one side thereof, an elastic membrane which is disposed between the pressure chamber and the micro drug chamber, and a laser unit which irradiates the pressure driving liquid stored in the pressure chamber with laser to generate bubble in the pressure driving liquid. The laser unit irradiates a laser beam having an oscillation wavelength range of 2.8 μm to 3.0 μm, and in particular, an Er:YAG laser having a wavelength of 2.94 μs (2940 nm) is used.

### TECHNICAL FIELD

The present invention relates to a transdermal drug delivery system of administering a drug solution through skin, and more particularly, a painless needle-free drug delivery system capable of delivering the drug solution into a human body or an animal body in a transdermal manner by administering the drug solution into skin tissue without perforation of a skin layer caused by a subcutaneous syringe needle by injecting the drug solution in a high-speed microjet form through a small nozzle by instantaneously exerting a strong force to the drug solution.

### BACKGROUND ART

In general, a variety of drug delivery systems have been applied as a method for parenterally administering a treatment drug solution into a patient's body in a medical field. In these drug delivery systems, the most commonly used method is a method using a needle-type syringe. In this method, a syringe having a syringe needle is pierced into a patient's skin, and a drug solution is directly injected. However, such a conventional injection method has a great shortcoming in that the patient suffers from an inconvenience of feeling a pain during the injection. In addition, the injection method has many problems such as a wound caused by perforation of a skin layer, a risk of secondary infection through the wound, and waste of resources due to difficulty in reusing the syringe.

Due to the shortcomings of the conventional needle-type syringe, development of needle-free drug delivery systems as a substitute for the needle-type syringe has been widely researched. In an attempt to develop the needle-free drug delivery system, there has been proposed a drug delivery system of ejecting a drug solution in a form of a microjet having a small diameter at a high speed and allowing the drug solution to be directly penetrated into an internal target spot through epidermis.

The research of such a microjet drug delivery system was first attempted in the 1930s. The initial microjet drug delivery system is a very basic drug delivery method using a simple microjet mechanism. The above microjet drug delivery system involves various problems in that there is a risk of cross infection, a splash back phenomenon occurs during the microjet injection, and an accurate penetration depth is difficult to adjust. Particularly, since such a conventional microjet drug delivery system still has a disadvantage in that the treatment is accompanied by a considerable pain, it was not widely adopted as a substitute for the existing syringe.

In addition, as a method for reducing the pain-related problem involved in the above microjet drug delivery system and stabilizing the drug administration, Stachowiak et al. have developed and proposed a microjet drug delivery system using a piezoelectric ceramic element (J.C. Stachowiak et al, Journal of Controlled Release 124: 88-97 (2009)). The microjet drug delivery system proposed by Stachowiak et al. is one in which a drug solution is injected at high speed in the form of a liquid microjet using vibration generated when an electric signal is applied to the piezoelectric ceramic element. According to the microjet drug delivery system to Stachowiak et al., the drug solution can be stably injected into the skin without touching the nervous tissues through a real-time change in injection speed of the microjet, thereby effectively reducing a pain during the treatment. However, the microjet control of a trace amount of the drug solution needs to be performed in order to implement the time-varying monitoring of the drug injection. The microjet drug delivery system using the piezoelectric ceramic element has a great difficulty in realizing an actual drug delivery system due to a limitation of microjet control precision.

Further, besides the above microjet drug delivery system using an electric element and device, according to a recent research result, a microjet drug delivery system using a laser beam was developed (V. Menezes, S. Kumar, and Takayama, Journal of Appl. Phys. 106, 086102 (2009)). In the aforementioned microjet drug delivery scheme using a laser beam, an aluminum foil is irradiated with a laser beam to generate a shock wave so that a drug solution is injected in the form of microjet. The microjet drug delivery scheme has an advantage in that the laser beam as high energy can be concentrated on a very small area, so that a precise level of needle-free drug delivery system can be implemented. However, the aforementioned microjet drug delivery system using the laser beam and the shock wave has problems in that it is impossible to inject continuously controlled microjet, and particularly in the aforementioned system, and since the used aluminum foil is deformed, it is impossible to re-use the used syringe.

Thus, in order to solve the problems of the drug delivery system of the related art, the inventor continued to study. As a result, the inventor developed a novel type of a needle-free drug delivery system of administering a drug solution into bodily tissue by injecting the drug solution in a high-speed microjet form by using elastic membrane and volume expansion of a liquid in a sealed pressure chamber due to generation of a bubble which is generated by irradiating the liquid in the sealed pressure chamber with a laser beam. Such a novel type of a microjet drug delivery system was filed as Korean Patent Application No. 10-2010-56637 (name of the invention: microjet drug delivery system).

The invention of the application previously filed by the inventor relates to a microjet drug delivery system of administering a drug solution by injecting the drug solution in a high-speed microjet form through a nozzle having a small diameter by applying pressure to the drug solution. The microjet drug delivery system has a configuration including a pressure chamber 10 having a predetermined which is hermetically filled with a pressure driving liquid, a drug chamber 20 which is disposed adjacent to the pressure chamber 10 to store a drug solution, an elastic membrane 30 which is disposed between the pressure chamber 10 and the drug chamber 20 to partition the pressure chamber and the drug chamber, and an energy focusing unit 40 which concentrates strong energy of a laser beam or the like on an inner portion of the pressure chamber 10.

According to the above-described microjet drug delivery system of the invention of the previously filed application, if the energy focusing unit 40 irradiates the pressure driving liquid 100 in the pressure chamber 10 with the strong energy of the laser beam or the like in a concentrated manner, a bubble is generated in the pressure driving liquid 100, and in the course of rapid expansion and disappearance of the generated bubble, the elastic membrane is expanded and vibrated. Due to the expansion and vibration of the elastic membrane, the drug solution in the drug chamber 20 is rapidly ejected through the nozzle, so that the microjet of the drug solution is injected at an enough speed to penetrate soft tissue of the body.

In the invention of the application previously filed by the inventor, a general Q-switched Nd:YAG laser apparatus widely used as a medical laser apparatus is used, and the laser apparatus outputs a laser beam having a wavelength range of 532 nm, a pulse period of 5 to 9, and a frequency of 10 Hz. However, as a result of further studies performed by the inventor, in a microjet drug delivery system using an Nd:YAG laser having the aforementioned wavelength range, although the speed of microjet injection is high, the penetration depth and diffusion area in the bodily tissue are insufficient, and there is a shortcomings in terms of the distribution of the penetrated drug solution in the tissue.

In particular, in the invention of the application previously filed by the inventor, as a result of analysis of pictures of injected microjets photographed by a high-speed camera, it was found that the progression of the microjet is not smooth and somewhat disturbed. The phenomenon is predicted to occur for the following reason. A second microjet is generated due to a shock wave after a first microjet according to the expansion of the bubble. The speed of some droplets in the second microjet is higher than the speed of the precedingly ejected drug solution, so that the phenomenon occurs in the course of the droplets of the second microjet colliding from behind and passing the precedingly ejected microjet.

Therefore, the inventor studied to solve the above-described problems of the microjet drug delivery system of the invention of the previously filed application and to develop more efficient drug delivery system. As a result, the below-described enhanced microjet drug delivery system was developed.

### DISCLOSURE

### Technical Problem

The present invention is to provide a drug delivery system capable of being used as a substitute for an existing needle-type syringe and capable of injecting a drug solution more safely, simply, and effectively without a pain caused by syringe injection by penetrating the drug solution into skin tissue without use of a syringe needle by injecting the drug solution in a form of microjet through laser irradiation.

In particular, the present invention is to further improve an existing microjet drug delivery system previously developed by the inventor described above and to provide a microjet drug delivery system capable of securing a deeper, wider penetration range with energy equal to or lower than that of the existing microjet drug delivery system in administration of the drug solution into bodily tissue.

### Technical Solution

According to an aspect of the present invention, there is provided a microjet drug delivery system including a pressure chamber which has a predetermined storing space and of which a closed inner portion is hermetically filled with water or a liquid material including water as a pressure driving liquid, a drug chamber which is disposed adjacent to the pressure chamber and is configured to store a drug solution in a predetermined storing space and where a micro nozzle ejecting the drug solution as a microjet to an outside is formed at one side thereof, an elastic membrane which is disposed between the pressure chamber and the micro drug chamber to partition the pressure chamber and the micro drug chamber, and a laser unit which irradiates the pressure driving liquid stored in the pressure chamber with laser to generate bubble in the pressure driving liquid.

In the microjet drug delivery system according to the present invention, the laser unit irradiates a laser beam having an oscillation wavelength range of 2.8 μm to 3.0 μm.

In particular, in the microjet drug delivery system according to the present invention, preferably, the laser unit is an Er:YAG laser oscillation apparatus irradiating a laser beam having a wavelength of 2.94 μm (2940 nm). More preferably, the laser unit irradiates a laser beam having a wavelength of 2.94 μm and a pulse duration of 200 to 300 μs.

### Advantageous Effects

According to a microjet drug delivery system having the above configuration according to the present invention, in the injection of the drug solution, instead of directly exerting an external force to a to-be-injected solution or performing other operations thereon, the microjet injection of the drug solution is performed by generating an instantaneous bubble by concentrating energy of a laser beam or the like on a separate pressure driving liquid and by using a deformation in an elastic membrane caused by expansion in volume and occurrence of a shock wave due to the generation and disappearance of the bubble, and thus, the high-speed microjet injection of the drug solution can be penetrated into a body through the skin tissue, so that it is possible to inject the drug solution effectively without a pain caused by a needle-type syringe.

In particular, according to the above-described microjet drug delivery system according to the present invention, in comparison with the existing Nd:YAG laser microjet drug delivery system, it is possible to secure a deeper, wider penetration range with energy equal to or lower than that of the existing microjet drug delivery system in administration of the drug solution into bodily tissue, so that it is possible to further improve efficiency and reliability of the microjet drug delivery system.

In addition, according to the above-described microjet drug delivery system according to the present invention, in comparison with the existing microjet drug delivery system, at the time of injection it is possible to reduce splash-back and reduce damage to the skin.

### BRIEF DESCRIPTION OF DRAWINGS

FIG. 1 illustrates diagrams of a basic configuration of a microjet drug delivery system according to the present invention and an operation mechanism of microjet injection of a drug solution.

FIGS. 2a and 2b are images obtained by sequentially photographing a bubble generated in a pressure driving liquid when an Nd:YAG laser beam and an Er:YAG laser beam are applied to a microjet injector for test.

FIGS. 3a and 3b are images obtained by sequentially photographing progression of microjet with a super-high-speed camera in an Er:YAG laser microjet drug delivery system according to the present invention and an Nd:YAG laser microjet drug delivery system according to an invention of a previously filed application.

FIG. 4 illustrates graphs of speeds of microjet generated by the Nd:YAG laser and the Er:YAG laser in the aforementioned microjet injector test.

FIG. 5 illustrates microjet powers calculated based on a predetermined equation in the Nd:YAG laser microjet drug delivery system and the Er:YAG laser microjet drug delivery system.

FIG. 6 illustrates graphs of amounts of administered drug solution per microjet in the Nd:YAG laser microjet drug delivery system and the Er:YAG laser microjet drug delivery system.

FIG. 7 illustrates progression distances of microjet where breakup of microjet occurs in the Nd:YAG laser microjet drug delivery system and the Er:YAG laser microjet drug delivery system.

FIG. 8 illustrates images of an experiment set used for an animal tissue experiment in the Nd:YAG laser microjet drug delivery system and the Er:YAG laser microjet drug delivery system.

FIG. 9 illustrates states of stained FITC according to penetration of an experimental drug solution into skin tissue as results of the animal tissue experiment.

FIG. 10 is an image illustrating a comparison of maximum sizes of the bubble generated in the Nd:YAG laser microjet drug delivery system and the Er:YAG laser microjet drug delivery system.

### BEST MODES FOR CARRYING OUT THE INVENTION

Hereinafter, a microjet drug delivery system according to the present invention will be described more in detail with reference to the attached drawings and data of experiment results.

In general, a “drug delivery system” collectively denotes a method of means for adjusting an ejection speed of a drug solution or effectively delivering the drug solution to a target spot in administration of the required drug solution into a body. In recent years, in the field of the drug delivery system, administration and delivery of a drug solution through skin by using skin tissue as a target spot have drawn much attention. As a representative one of the transdermal drug delivery systems using skin, there is a drug solution patch.

A skin tissue is mainly configured with epidermis and dermis. The epidermis is the outermost layer of skin. The epidermis has a function as a protective layer of preventing infiltration of external harmful pathogenic bacteria and a function of preventing moisture from evaporating from a body. Although a thickness of the epidermis varies with age, gender, and the like, the thickness of a human is about 500 μm.

The epidermis has a very important role in the implementation of a painless drug delivery system. Since almost no veins and nerve cells exist in the epidermis, if the drug solution is penetrated through the epidermal cells to the depth of the upper portion of the dermis in the transdermal administration of the drug solution through the skin, it is possible to implement an effective drug delivery system while minimizing bleeding and pain occurring according to the skin-penetrating administration of the drug solution.

Therefore, as described below, a microjet drug delivery system according to the present invention relates to a technique of transdermal delivery of a drug solution through skin. Since drug microjet basically has a speed enough to penetrate through epidermis of the skin into dermis, appropriate performance as a painless transdermal drug delivery system can be secured, the penetrated drug solution can be uniformly diffused into a tissue, and a splash-back phenomenon can be minimized. Therefore, in comparison to existing microjet drug delivery systems, it is possible to efficiency and reliability.

Hereinafter, a main technical configuration of an enhanced microjet drug delivery system according to the present invention will be described more in detail, and improved effects of the present invention will be described with respect to experiment results in comparison to the existing systems.

[Basic Configuration and Operation Principle of Microjet Drug Delivery System According to the Invention]

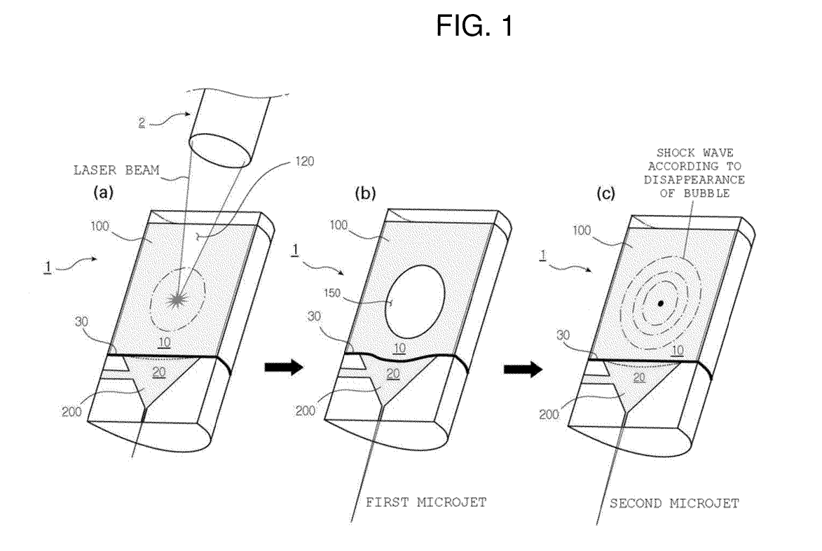

FIG. 1 illustrates a basic configuration of the microjet drug delivery system according to the present invention and an operation mechanism of microjet injection of a drug solution. As illustrated in (a) to (c) of FIG. 1, the microjet drug delivery system is configured to include a microjet injector unit 1 as a syringe which stores a predetermined amount of the drug solution and administers the drug solution into a body by microjet injection and a laser unit 2 as means for supplying driving energy for the microjet injection of the drug solution in the microjet injector unit 1.

As illustrated, the microjet injector unit 1 has an overall structure where two chambers are formed to be consecutive in one housing. A drug chamber 20 which stores a to-be-injected drug solution 200 is disposed at the front surface side of the microjet injector unit, and a pressure chamber 10 which is a pressure chamber for applying a driving force to the drug solution 200 of the drug chamber 20 and of which inner portion is hermetically filled with a pressure driving liquid 100 is disposed at the rear surface side of the microjet injector unit to be consecutive to the drug chamber. As a partition wall which partitions the drug chamber 20 and the pressure chamber 10, an elastic membrane 30 is formed with an elastic material. The elastic membrane is configured to be elastically expanded and deformed according to a change in physical state of the pressure driving liquid 100 of the pressure chamber 10 to apply pressure to the drug solution 200 of the adjacent drug chamber 20, so that the drug solution can be ejected.

In the above-described microjet drug delivery system according to the present invention, as the pressure driving liquid 100, various liquid materials such as liquid, sol, or gel capable of generating bubble by receiving laser energy from the laser unit 2 may be used. Most preferably, water may be used. The Er:YAG laser which is one of the main characteristic components of the present invention has high absorbance with respect to water. In case of using the water, bubble can be very efficiently generated according to absorption of the laser energy. In addition, in the present invention, although a single substance of water may be used as the pressure driving liquid 100, a mixture of the water with other kinds of liquid (for example, alcohol) or a liquid material may be used. An aqueous solution where other solids are dissolved may also be used.

Furthermore, in case of using water as the pressure driving liquid 100, it is preferable that degassed water be used so as to minimize deterioration in injection efficiency caused by residual bubble before and after the laser irradiation and the injection. In particular, if a water-soluble electrolyte (for example, salt) is added to pure water, the energy required for destructing the structure of the liquid becomes low due to the effect of ionization of molecules, the efficiency is further improved.

The elastic membrane 30 is a thin membrane member made of an elastic material such as a natural rubber or synthetic rubber. If physical pressure is exerted from an outside, the elastic membrane is deformed from a tensely stretched state, and after that, the elastic membrane is elastically recovered from the deformed state. As a material used for the elastic membrane 30, preferably, a nitril butadiene rubber (NBR) may be used. Since the NBR has good elasticity and low thermal conductivity, damage to the drug solution caused by heat generated during the laser irradiation can also be prevented.

The laser unit 2 is a laser oscillation apparatus generating a laser beam. In the present invention, the laser beam emitted from the laser unit 2 is focused and irradiated on one spot in the pressure driving liquid 100 of the pressure chamber 10, so that a bubble is generated.

Although, in the invention of the application previously filed by the inventor, a Q-switched Nd:YAG laser apparatus widely used as a medical laser apparatus is used as the most preferable apparatus of the laser unit, in the present invention, an Er:YAG laser apparatus having a wavelength range of 2.9 μm is used as a laser generating apparatus selected by a result of various researches and experiments. In this case, it is found that the Er:YAG laser has much better effects than the existing Nd:YAG laser in terms of penetration depth of microjet and distribution state of microjet.

In the microjet drug delivery system having the above-described configuration according to the invention, the driving force for the microjet injector of the drug solution 200 is generated from the pressure driving liquid 100 filling the pressure chamber 10. In the present invention, energy is applied so as to be concentrated on the pressure driving liquid 100, so that the bubble (150) is rapidly generated in an inner portion of the liquid. Due to the generation of the bubble, the elastic membrane 30 is instantaneously, strongly biased toward the drug chamber, so that the driving pressure is applied to the to-be-injected drug solution 200 in the drug chamber 20.

As illustrated in (a) of FIG. 1, if the laser unit 2 is driven to irradiate the focused portion of the pressure driving liquid 100 hermetically filling the pressure chamber with a laser beam, the molecular structure of the pressure driving liquid 100 is destructed due the concentrated energy of the laser beam, so that a gas bubble 150 is generated in the liquid as illustrated in (b) of FIG. 1.

As described above, the bubble generated in the pressure driving liquid 100 is instantaneously, rapidly expanded and disappears. The rapid change in volume caused by the rapid expansion and disappearance of the bubble in the sealed pressure chamber 10 causes a deformation of the elastic membrane 30. Due to the deformation of the elastic membrane, an external force is exerted on the drug solution 200 in the drug chamber 20 adjacent to the elastic membrane, so that the drug solution is strongly, rapidly ejected through the micro nozzle. Therefore, the drug microjet having an enough speed to penetrate into the skin tissue is generated.

As illustrated in (c) of FIG. 1, in some speed of expansion and disappearance of the bubble, a shock wave may be generated due to the rapid change in volume at the time of disappearance of the bubble. The generated shock wave is transferred to the elastic membrane to generate vibration of the elastic membrane, so that a second microjet may be generated.

[Comparison of Specific Operation Mechanism]

As described above, unlike the microjet drug delivery system of the invention of the application previously filed by the inventor where a Q-switched Nd:YAG laser is used as the laser generation unit, in the present invention, an Er:YAG laser is used so as to further improve efficiency and reliability.

As described above, basically, the Nd:YAG laser microjet drug delivery system of the invention of the previously filed application and the Er:YAG laser microjet drug delivery system according to the present invention has the same configuration in that the microjet injection of the drug solution is performed by the vibration of the elastic membrane caused by the bubble generated by applying the laser beam to the liquid 100 in the pressure chamber 10 including the elastic membrane 30 at one side. The two systems are different in terms of specific operation mechanism of generation and growth of the bubble through the more in-depth studies.

Namely, in the Nd:YAG laser microjet drug delivery system, the bubble is generated by cavitation, and the bubble is grown by influence of a pressure gradient. In contrast, in the Er:YAG laser microjet drug delivery system according to the present invention, the bubble is generated by boiling of the liquid absorbing the laser energy, and the bubble is grown by influence of a temperature gradient.

First, the mechanism in the Nd:YAG laser microjet drug delivery system will be described in detail. The main factor for the generation of the bubble is cavitation. Namely, if strong energy is concentrated on a spot where the laser beam is focused, the bonding of water molecules are locally destructed (optical breakdown), and the cohesion force becomes weak. The liquid pressure is decreased down to the saturated water vapor pressure or less, so that the gas bubble is generated. According to the inventor's experiment and observation, it is found that the gas bubble exists mainly in a plasma state due to strong energy of a short wavelength Nd:YAG laser.

In contrast, in the Er:YAG laser microjet drug delivery system according to the present invention, since the Er:YAG laser is a laser having a wavelength range which is well absorbed by water, the temperature of the liquid where the energy is absorbed in the focus portion and the laser beam path around the focus portion is increased up to the boiling point or more, so that evaporation occurs, and thus, the vaporized gas bubble is generated.

As described above, the existing Nd:YAG laser microjet drug delivery system the Er:YAG laser microjet drug delivery system according to the present invention are different in terms of bubble generation mechanisms of cavitation and boiling. The systems are also different in terms of shape, size, growth speed, and retention time of the bubble.

In terms of the influence of the pulse duration of the oscillation laser, in the Nd:YAG laser microjet drug delivery system according to the invention of the previously filed application, since the bubble is generated by the cavitation, the retention time of the bubble is short, and a laser beam is applied for a relatively short pulse period, so that the generation and disappearance of the bubble are repeated for a very short time. According to the Nd:YAG laser microjet drug delivery system, since the generation, expansion, and disappearance of the bubble are rapidly repeated, a shock wave is generated within the liquid. The shock wave causes vibration of the elastic membrane, and it is found that the shock wave functions as a main driving force for microjet injection of the drug solution. In the Nd:YAG laser microjet drug delivery system according to the invention of the previously filed application, it is found that the microjet injection of the drug solution is performed by two functions, that is, the increase in volume of the pressure driving liquid caused by the generation of the bubble and the vibration of the elastic membrane caused by the shock wave.

In contrast, in the Er:YAG laser microjet drug delivery system according to the invention, the bubble is generated by boiling of the liquid absorbing laser energy. Although the speed of expansion after the generation of the bubble is slow, the size of the bubble is much larger than that of the case of using the Nd:YAG laser, and the retention time thereof is also long. In addition, in the Er:YAG laser microjet drug delivery system according to the invention, the meaningful shock wave is not generated at the time of expansion of the bubble. Since the elastic membrane is mainly expanded according to the volume expansion of the bubble, the microjet injection is performed by a single function of exerting a force on the drug solution.

Therefore, due to the difference in the basic mechanism, the existing Nd:YAG laser microjet drug delivery system and the Er:YAG laser microjet drug delivery system according to the present invention are different in terms of characteristics of the microjet drug delivery system such as shape of generated bubble, microjet injection efficiency, and injection performance. Hereinafter, the performance and characteristics the two systems are compared through experiments.

[Manufacturing of Test Product and Comparison Test]

In order to evaluate the performance and enhanced effects of the above-described microjet drug delivery system according to the invention, a microjet drug delivery system is implemented by using test products, and the test is performed.

The body of the microjet injector for test is made of stainless steel and has a shape of a cylinder. The microjet injector is assembled by disposing the elastic membrane between two hollow stainless steel cylinders and by fastening the cylinders by a ring screw. The micro nozzle of the distal end has a diameter of 100 μm. For the elastic membrane, nitril butadiene rubber (NBR) having a thickness of 200 μm, hardness of 53, limit strength of 101.39 kg/cm², and an expansion ratio of 449.79% is used.

For the comparison with the existing microjet drug delivery system, the experiment is performed by alternately using the Nd:YAG laser oscillation apparatus and the Er:YAG laser oscillation apparatus as the laser unit for the microjet injector for test. As the existing Nd:YAG laser apparatus, a Q-switched Nd:YAG laser apparatus widely used as a medical laser apparatus is used. The above-described Nd:YAG laser apparatus outputs a laser having a wavelength of 1064 nm, a pulse duration of 7 ns, and output energy of 408 mJ/pulse.

The Er:YAG laser apparatus according to the invention outputs a laser having an oscillation wavelength of 2.94 μm (2940 nm), a pulse duration of 250 μs, and output energy of 408 mJ/pulse. Table 1 lists laser output characteristics of the Nd:YAG laser microjet drug delivery system and the Er:YAG laser microjet drug delivery system used for the test. In the Er:YAG laser microjet drug delivery system according to the present invention, the pulse duration is set by considering the expansion time and disappearance time of the bubble. In the case where the pulse duration is 150 μs or less, the bubble is not generated well. In the case where the pulse duration is 500 μs or more, the bubble is grown too slowly, and the speed and pressure of microjet is not sufficiently strong. As a result of repeated experiments, a good result is obtained in a pulse duration of 200 to 300 μs.

TABLE 1
Information on Output of Laser of Systems

| | Nd:YAG | | Er:YAG | |
|---|---|---|---|---|
| Laser Energy | 408 | mJ | 408 | mJ |
| Wavelength | 1064 | nm | 2940 | nm |
| Pulse Duration | 7 | ns | 250 | μs |

In the above-described microjet injector for test, as pressure driving liquid, water (distilled water) is used, and 3% salt is dissolved as an electrolyte in the degassed water.

[Result of Test]

1. Comparison of Shape of Generated Bubble

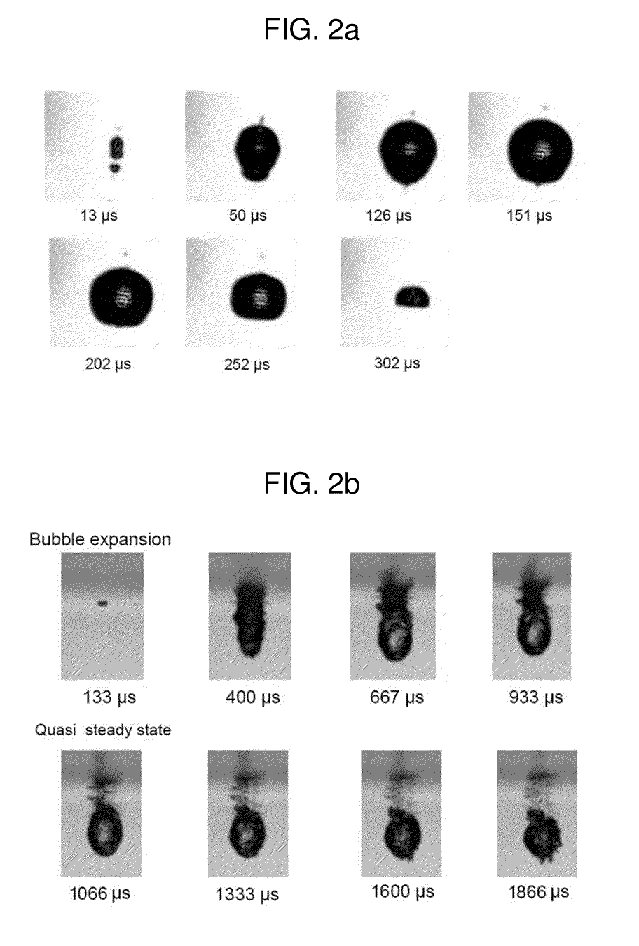

FIGS. 2a and 2b are images obtained by sequentially photographing from generation to disappearance of a bubble in a pressure driving liquid when an Nd:YAG laser beam and an Er:YAG laser beam are applied to the above-described microjet injector for test. FIG. 2a is an image obtained by photographing the bubble generated in the existing Nd:YAG laser microjet drug delivery system, and FIG. 2b is an image obtained by photographing the bubble generated in the Er:YAG laser microjet drug delivery system according to the present invention.

As illustrated in FIG. 2a, in the existing Nd:YAG laser microjet drug delivery system, the bubble is expanded to its maximum size within a very short time (151 μs), and its maximum diameter is 3216 μm

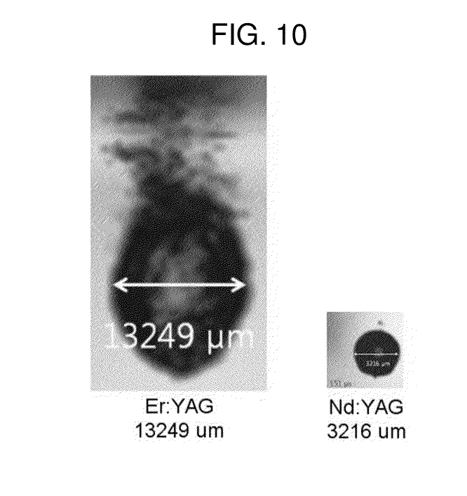

In contrast, as illustrated in FIG. 2b, in the Er:YAG laser microjet drug delivery system according to the present invention, the bubble is expanded for a relatively long time (933 μs) after the generation of the bubble. In the Er:YAG laser microjet drug delivery system, the maximum diameter of the bubble is 13249 μm, which is much larger than that of the Nd:YAG laser microjet drug delivery system (refer to comparison of images in FIG. 10). As seen from the results illustrated in FIGS. 2a and 2b and FIG. 10, it can be expected that, in comparison with the existing Nd:YAG laser microjet drug delivery system, the Er:YAG laser microjet drug delivery system according to the present invention can efficiently generate a large amount of drug microjet.

With respect to the shape of the generated bubble, as illustrated in FIG. 2a, the bubble in the Nd:YAG laser microjet drug delivery system has a cross section of a substantial circle, and in contrast, as illustrated in FIG. 2b, the bubble in the Er:YAG laser microjet drug delivery system according to the present invention has a cross section of a vertically elongated ellipse. The reason is as follows. In the case of the Nd:YAG laser, the energy of the laser beam having a wavelength range which is almost not absorbed by water is concentrated on the focus portion, and in this portion, the bonding structure of water molecules is destructed. In the case of the Er:YAG laser, the energy of the laser beam having a wavelength range which is well absorbed by water is absorbed by water in the laser beam path (denoted by reference numeral 120 in FIG. 1 (a)) as well as the focus portion, so that evaporation occurs in the upper portion of the focus portion, and thus, the vertically elongated bubble is generated.

TABLE 2
Comparison of Bubble Characteristics Between
Two System

| | Nd:YAG | | Er:YAG | |
|---|---|---|---|---|
| Growth Time of | 151 | μs | 933 | μs |
| Bubble | | | | |
| Maximum | 3.2 | mm | 13.2 | mm |
| Diameter | | | | |
| Maximum | 39 | m/s | 26.5 | m/s |
| Expansion Speed | | | | |

| | | |
|---|---|---|
| Smoothness of | Smooth | Relatively Rough |
| Surface | | |
| Shape of Bubble | Spherical Shape | Vertically Elongated |
| | | Shape |
| Progression of | Expansion | Disappearance in Quasi- |
| Disappearance | Disappearance | Steady State After |
| of Bubble | Rebounding | Expansion |

2. Comparison of Microjet Power

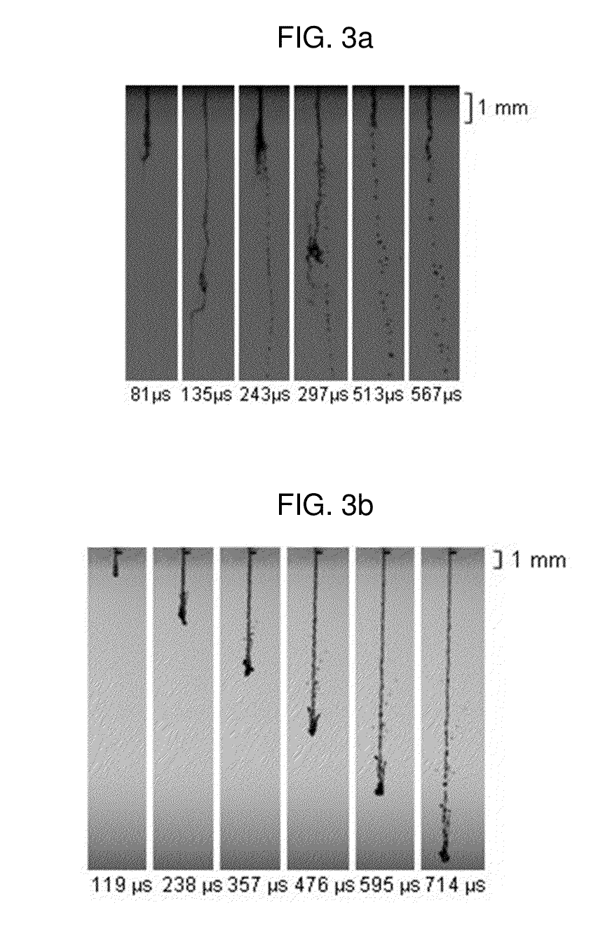

FIGS. 3a and. 3b illustrate images obtained by sequentially photographing progression of microjet with a supper-high-speed camera in the Er:YAG laser microjet drug delivery system according to the present invention and the existing Nd:YAG laser microjet drug delivery system of the invention of the previously filed application. FIG. 3a illustrates an image of the microjet in the existing Nd:YAG laser microjet drug delivery system, and FIG. 3b illustrates an image of the microjet in the Er:YAG laser microjet drug delivery system according to the present invention.

As illustrated in FIG. 3a, in the existing Nd:YAG laser microjet drug delivery system, the shape of the progression of the drug microjet is not smooth, and bad jet stability such as an irregularly disturbed shape of the drug solution is observed at the distal end of the microjet. In particular, as illustrated in FIG. 3a, the drug microjet is not injected at one time in a concentrated manner, but the drug microjet is injected at two times in a divided manner, so that it is predicted that there is a loss in the driving force and the efficiency.

In contrast, as illustrated in FIG. 3b, in the Er:YAG laser microjet drug delivery system according to the present invention, the shape of the progression of the drug microjet is much more smooth and uniform without an irregularly disturbed shape than the shape of the existing microjet drug delivery system illustrated in FIG. 3a. It can be seen that, the drug microjet is injected at one time in a concentrated manner, so that the good stability of shape of microjet is obtained.

2. Comparison of Speed and Shape of Microjet

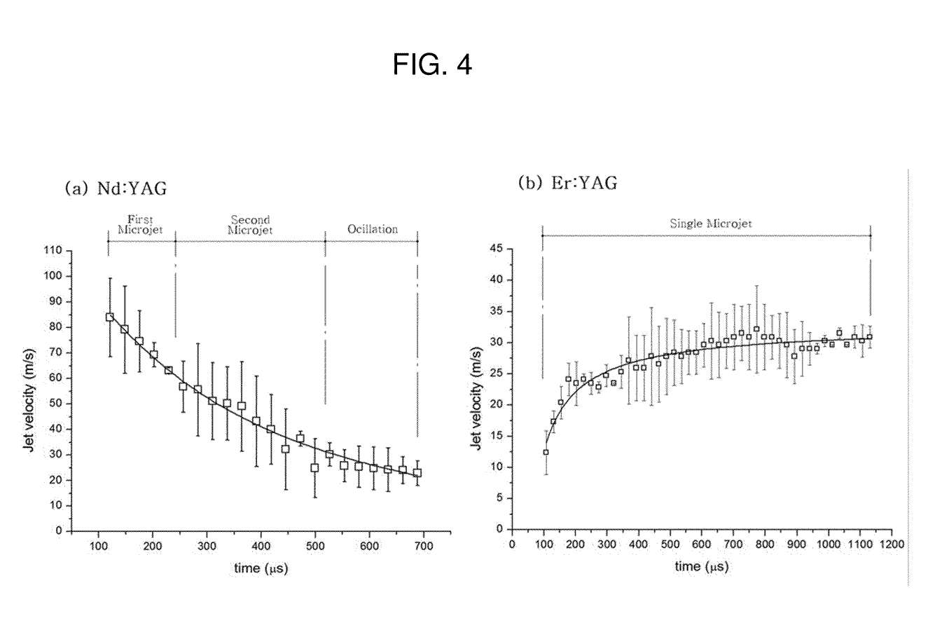

FIG. 4 illustrates time-varying graphs of speeds of microjet generated by the Nd:YAG laser microjet drug delivery system and the Er:YAG laser microjet drug delivery system in the aforementioned microjet injector test. (a) of FIG. 4 illustrates a change in speed of microjet generated by the Nd:YAG laser microjet drug delivery system, and (b) of FIG. 4 illustrates a change in speed of microjet generated by the Er:YAG laser microjet drug delivery system.

As seen from the resulting graphs of FIG. 4, the initial speed of microjet in the Nd:YAG laser microjet drug delivery system is higher than that in the Er:YAG laser microjet drug delivery system. As seen from (a) of FIG. 4, with respect to the change in speed of microjet, in the Nd:YAG laser microjet drug delivery system, the maximum speed appears at the initial injection of the microjet, and after that, the speed of microjet is gradually decreased.

In contrast, as seen from (b) of FIG. 4, in the Er:YAG laser microjet drug delivery system, the initial speed of microjet is low, and after that, the speed of microjet is rapidly increased. After the speed of microjet reaches the maximum speed (about 50 m/s), the speed is maintained for a relatively long time.

The characteristic in injection shape of microjet is described more in detail. As illustrated in FIG. 3a and (a) of FIG. 4, in the Nd:YAG laser microjet drug delivery system, mainly two microjets are consecutively injected. In contrast, as illustrated in FIG. 3b and (b) of FIG. 4, in the Er:YAG laser microjet drug delivery system, the drug solution is continuously injected as a single microjet.

With respect to the retention time of microjet, in the Nd:YAG laser microjet drug delivery system, as described above, the first microjet of the two injected microjets is measured to have a retention time of 162±27 μs, and the second microjet is measured to have a retention time of 261±41 μs, so that a total of the retention times is 423±56 μs is obtained.

In contrast, in the Er:YAG laser microjet drug delivery system, the single injected microjet is measured to have a retention time of 940±50 μs, which denotes that the drug solution is ejected for a relatively long time.

TABLE 3
retention time of Microjet

| Nd:YAG | | Er:YAG | |
|---|---|---|---|
| First Microjet | 162 ± 27 μs | Single | 940 ± 50 μs |
| | | Microjet | |
| Second | 261 ± 41 μs | | |
| Microjet | | | |
| Total | 423 ± 56 μs | | |

3. Evaluation and Comparison of Injection Performance: Microjet Power and Penetration Performance

In order to evaluate the drug injection performance as a transdermal drug delivery system, 7% gelatin is used as a target model simulating skin tissue, and penetration performance to the gelatin is tested for the Nd:YAG laser microjet drug delivery system and the Er:YAG laser microjet drug delivery system.

As a preliminary stage for the penetration performance test, since the penetration depth of the drug solution into skin in the microjet drug delivery system is predicted to have a direct relation with the microjet power of the injected microjet, the microjet powers of the Nd:YAG laser microjet drug delivery system and the Er:YAG laser microjet drug delivery system are calculated based on the above-described test result of the relationship between the speed and time of the microjet. The microjet power is calculated by using the following equation.

P₀=½\dot{m}u₀²=⅛πρD₀²u₀³
P₀: MicrojetPower
\dot{m}: MassFlowRate
u₀: EjectionSpeed
D₀: NozzleDiameter

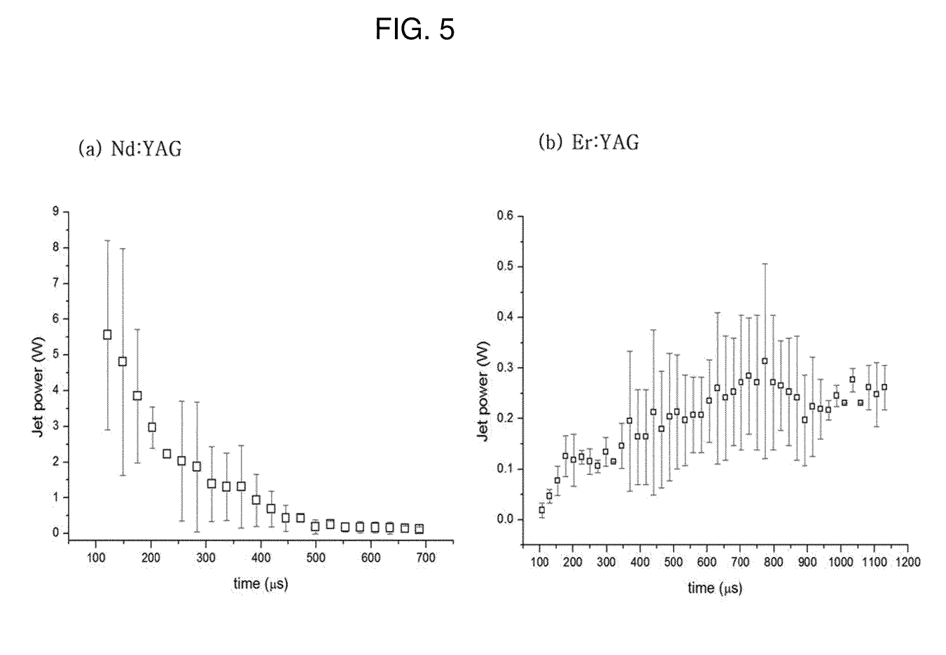

As seen from the above equation, the microjet power is proportional to the cube of the ejection speed. FIG. 5 illustrates graphs of microjet powers calculated based on the above equation in the Nd:YAG laser microjet drug delivery system and the Er:YAG laser microjet drug delivery system.

As seen from the graphs of FIG. 5, the peak power of the Nd:YAG laser microjet drug delivery system is much higher than that of the Er:YAG laser microjet drug delivery system, so that it is predicted that the skin penetration performance of the Nd:YAG laser microjet drug delivery system is better. However, as illustrated in FIG. 5, in the Nd:YAG laser microjet drug delivery system, the maximum instantaneous speed appears just after the microjet injection, and after that, the speed is rapidly decreased. In contrast, in the Er:YAG laser microjet drug delivery system, the initial speed is relatively low, but the speed is increased as the microjet injection progresses, so that the high speed is maintained for a relatively long time. The skin tissue penetration performance as a drug delivery system needs to be compared in various aspects, but the performance evaluation based on only the maximum instantaneous speed is not appropriate.

Namely, the important evaluation factor of the performance in the transdermal drug delivery system administering the drug solution in the body through skin is whether to efficiently, stably supply a required amount of the drug solution into the body. Therefore, in the microjet drug delivery system according to the present invention, in order to accurately evaluate the performance as the transdermal drug delivery system, whether the drug solution is penetrated in to the target tissue by a sufficient depth and whether a stable amount of administered drug solution can be secured are considered to be important evaluation factors. The experiment is performed, and the result is as follows.

4. Evaluation of Injection Performance: Comparison of Penetration Depth of Drug Solution

As described, penetration test is performed on 7% gelatin as a target model simulating skin tissue, and the penetration depth of the drug microjet into the gelatin is measured in the Nd:YAG laser microjet drug delivery system and the Er:YAG laser microjet drug delivery system. The result of the test is an average value of values obtained by the five-time measurement. In the Nd:YAG laser microjet drug delivery system, the average penetration depth is is 1.78 mm; and in the Er:YAG laser microjet drug delivery system, the average penetration depth is 1.66 mm.

As seen from the measurement results of the penetration depth, although the peak power of the microjet in the Er:YAG laser microjet drug delivery system is lower than that in the Nd:YAG laser microjet drug delivery system, irrespective of using much lower energy the penetration depth in the Er:YAG laser microjet drug delivery system is substantially equal to that in the Nd:YAG laser microjet drug delivery system.

5. Evaluation of Injection Performance: Comparison of Amount of Administered Drug Solution

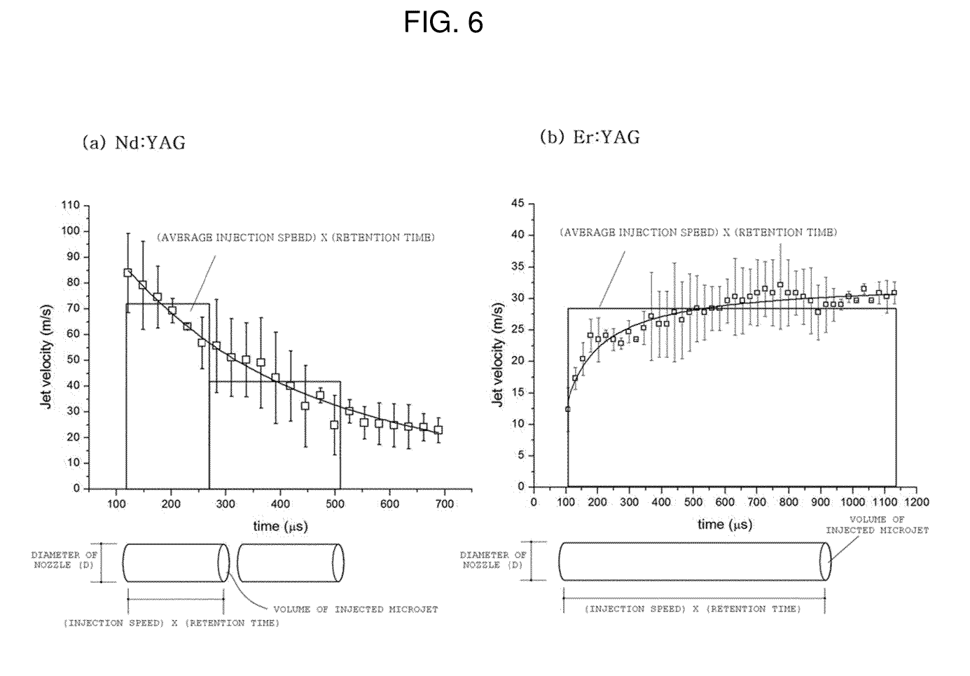

The amount of administered drug solution is evaluated as another evaluation factor of the injection performance as the microjet drug delivery system. The amounts of administered drug solution per microjet in the Nd:YAG laser microjet drug delivery system and the Er:YAG laser microjet drug delivery system are calculated based on the result of the relationship between the speed and time of the microjet illustrated in FIG. 3 (refer to FIG. 6).

As described above, in the Nd:YAG laser microjet drug delivery system, the two microjets are injected in a divided manner, and the injection amounts of the first and second microjet are individually calculated and summed up. In the Er:YAG laser microjet drug delivery system, since the single microjet is injected, and the injection amount of the single microjet is calculated. As illustrated in FIG. 6, the injection volume is calculated as a product of an average speed value (for values of time sections) and a retention time of microjet multiplied with a cross section area of a nozzle. The result is listed in the following Table 4.

TABLE 4
Comparison of Injection Volume of Microjet

| Nd:YAG | | Er:YAG | |
|---|---|---|---|
| Volume of | 143 ± 38 nL | Volume of | 416 ± 86 nL |
| First Microjet | | Single | |
| | | Microjet | |
| Volume of | 134 ± 27 nL | | |
| Second | | | |
| Microjet | | | |
| Total | 277 ± 65 nL | | |

As listed in the above result, the injection amount of the drug solution in the Er:YAG laser microjet drug delivery system is much larger than that in the Nd:YAG laser microjet drug delivery system. Therefore, in comparison with the existing Nd:YAG laser microjet drug delivery system, the Er:YAG laser microjet drug delivery system according to the present invention can secure a larger amount of administered drug solution irrespective of using the same energy, so that it is possible to implement a more efficient drug delivery system.

6. Comparison of Stability of Microjet

Another important evaluation factor of performance of the microjet drug delivery system is the stability of microjet. The stabilities of microjet in the Nd:YAG laser microjet drug delivery system and the Er:YAG laser microjet drug delivery system according to the present invention are compared, and the comparison results are as follows.

In a flow of a fluid, an a reference value for evaluation of stability, there is a Reynolds number. The following table lists values of Reynolds number calculated according to the Reynolds number equation in the Nd:YAG laser microjet drug delivery system and the Er:YAG laser microjet drug delivery system according to the present invention and values of Weber number in order to evaluate surface tension and characteristic of spraying.

TABLE 5
Reynolds Number in Nd:YAG Laser Microjet Drug Delivery
System

| | Reynolds Number | Weber Number | Weber Number of |
|---|---|---|---|
| Nd:YAG | (ReL) | (WeL) | Gas (Weg) |
| First | 11078 | 11386 | 13.5 |
| Microjet | | | |
| Second | 7081 | 6580 | 7.8 |
| Microjet | | | |

TABLE 6
Reynolds Number in Er:YAG Laser Microjet Drug Delivery
System

| | Reynolds Number | Weber Number | Weber Number of |
|---|---|---|---|
| Er:YAG | (ReL) | (WeL) | Gas (Weg) |
| Single | 4117 | 1572 | 1.9 |
| Microjet | | | |

As understood from the values of Reynolds number in the above tables, the microjet in the Nd:YAG laser microjet drug delivery system is in a completely turbulent flow state. In contrast, the microjet in the Er:YAG laser microjet drug delivery system according to the present invention is in a transition state between a laminar flow state and a turbulent flow state. Accordingly, it is numerically evaluated that the Er:YAG laser microjet drug delivery system according to the invention has much better stability in the progression of the microjet (standard of evaluation of transition state: 103<Re<104)

As another evaluation factor of the stability of microjet, there is a breakup time which is taken from the generation of microjet and the breakup. Namely, as the breakup time is lower, the microjet progresses in the turbulent flow according to the increase in the speed of microjet, so that the microjet is likely to be more stable.

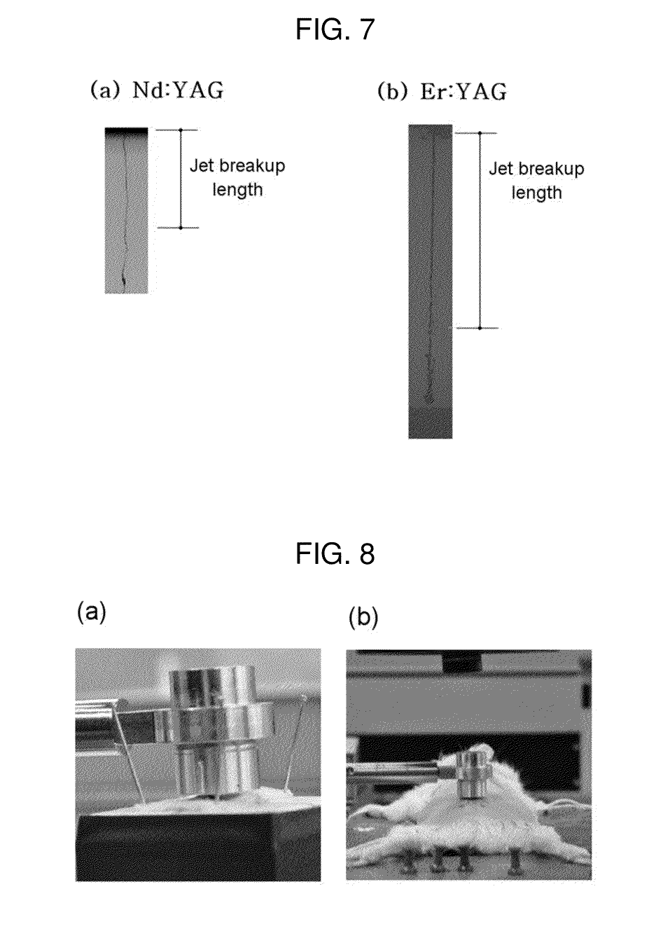

A method of evaluation of the breakup time of microjet is to analyze and evaluate pictures obtained by photographing the microjets injected in the systems with a super-high-speed camera (refer to FIG. 7). More specifically, a time when the breakup of microjet occurs after the microjet injection and an injection distance are measured as evaluation factors. The measurement results of the systems are listed in the following Tables 7 and 8.

TABLE 7
Breakup Length and Time of Microjet in Nd:YAG Laser
Microjet Drug Delivery System

| | Breakup Length | Breakup Time of |
|---|---|---|
| Nd:YAG | (L/D) of Microjet | Microjet |
| First Microjet | 36.9 ± 8.9 | 135 ± 27 μs |
| Second Microjet | 23.5 ± 5.8 | 180 ± 31 μs |

TABLE 8
Breakup Length and Time of Microjet in Er:YAG Laser
Microjet Drug Delivery System

| | Breakup Length | Breakup Time of |
|---|---|---|
| Nd:YAG | (L/D) of Microjet | Microjet |
| Single Microjet | 67.1 ± 0.4 | 678 ± 17 μs |

As understood from the result in the above table, in comparison with the Nd:YAG laser microjet drug delivery system, in the Er:YAG laser microjet drug delivery system, disturbance of the microjet does not occur after the microjet injection, and a good result can be obtained in terms of the shape retention time and distance of the microjet. Therefore, in comparison with the existing Nd:YAG laser microjet drug delivery system, in the Er:YAG laser microjet drug delivery system according to the present invention, the microjet can be more stably injected, it is possible to greatly reduce the splash-back phenomenon in the case of use for a target spot and to accurately, effectively administer the drug solution into the skin tissue.

[Penetration Experiment for Animal Tissue and Results]

Next, in order to evaluate actual drug administration performance of the existing Nd:YAG laser microjet drug delivery system and the Er:YAG laser microjet drug delivery system according to the present invention, a drug administration experiment is performed on animal tissue.

As a sample of the experiment for animal, a five-week-old guinea-pig is used. One day before the experiment, hair is removed clearly from an abdominal area and a back area of a guinea-pig by using a wax, and sterilization by a phosphate buffered saline (PBS) solution is performed. After that, the experiment is performed.

As a drug solution for penetration, used is a solution obtained by dissolving 0.1 mg/ml biotin and fluorescein isothiocyanate (FITC) as a fluorescent substance at a concentration of 0.05 mg/ml in a dimethyl sulfoxide (DMSO) solution. FIG. 8 illustrates images of an experiment set used for the animal tissue experiment. (a) of FIG. 8 illustrates a penetration experiment set for the abdominal area of the guinea-pig in the Nd:YAG laser microjet drug delivery system, and (b) of FIG. 8 illustrates a penetration experiment set for the back area of the guinea-pig in the Er:YAG laser microjet drug delivery system according to the present invention.

TABLE 9
Comparison of Characteristics of Lasers Used in Animal
Experiment

| | Nd:YAG | | Er:YAG | |
|---|---|---|---|---|
| Laser Energy | 2.7 | J | 1.57 | J |
| Wavelength | 1064 | nm | 2940 | nm |
| Pulse Duration | 7 | ns | 250 | μs |

| | | |
|---|---|---|
| Target Spot | Abdominal Tissue | Back Tissue |

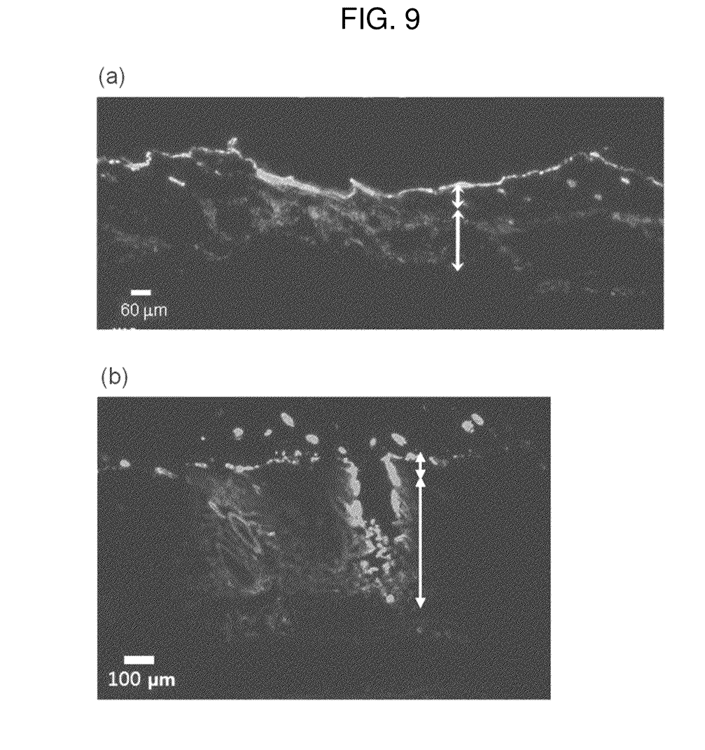

FIG. 9 illustrates states of stained FITC according to penetration of an experimental drug solution into skin tissue as the results of the animal tissue experiment. (a) of FIG. 9 illustrates a fluorescence image of a cross section of the abdominal tissue of the guinea-pig where the experimental drug solution is penetrated by the Nd:YAG laser microjet drug delivery system, and (b) of FIG. 9 illustrates a fluorescence image of a cross section of the back tissue of the guinea-pig where the experimental drug solution is penetrated by the Er:YAG laser microjet drug delivery system according to the present invention.

As understood from the images of FIG. 9, in both systems, the drug solution is penetrated through the epidermis of the living tissue into the dermis by a sufficient depth, so that both systems have appropriate performance as the transdermal drug delivery system.

In particular, as understood from (a) of FIG. 9, in comparison with the existing Nd:YAG laser microjet drug delivery system, the Er:YAG laser microjet drug delivery system according to the present invention can penetrate the drug solution into the skin tissue by a sufficient depth (about 450 μm) by using much lower output energy, it is possible to further improve the efficiency.

In addition, with respect to the concentration of the penetrated drug solution, in the existing Nd:YAG laser microjet drug delivery system, the penetrated drug solution is mainly concentrated on the epidermis side, and as the depth is increased, the concentration is low. In contrast, the Er:YAG laser microjet drug delivery system according to the present invention has a sufficient injection volume, and thus, a good penetration concentration can be obtained at a deep depth.

Accordingly, in comparison with the existing Nd:YAG laser microjet drug delivery system, the Er:YAG laser microjet drug delivery system according to the present invention can administer a larger amount of the drug solution into the bodily tissue by a sufficient depth by using energy equal to or lower than that of the existing system, so that it is possible to further improve the efficiency and to obtain much better performance in terms of uniformity of diffusion of the drug solution and reduction of the splash-back phenomenon. Since the microjet drug delivery system according to the present invention has very appropriate performance as a transdermal drug delivery system of delivering a drug solution through a skin layer, the microjet drug delivery system can be used as a very preferable microjet drug delivery system of administering various types of drug solutions such as treatment drug solutions, cosmetic emulsions, anesthetics, hormone drugs, and vaccines into a body in various fields such as a medical field, a cosmetic field, and a livestock field.

While the present invention has been particularly shown and described with reference to exemplary embodiments thereof, it will be understood by those skilled in the art that various changes in form and details may be made therein without departing from the spirit and scope of the present invention as defined by the appended claims.

### INDUSTRIAL APPLICABILITY

A microjet drug delivery system according to the present invention can be very used as a useful transdermal drug delivery system of delivering a drug solution through a skin layer basically in a medical field and as a very preferable tool of administering various types of drug solutions such as treatment drug solutions, cosmetic emulsions, anesthetics, hormone drugs, vaccines, and nutritional supplements into a body in various other fields such as a cosmetic field and a livestock field.

1. A microjet drug delivery system comprising:
a pressure chamber which has a predetermined storing space and of which a closed inner portion is hermetically filled with water or a liquid material including water as a pressure driving liquid;
a drug chamber which is disposed adjacent to the pressure chamber and is configured to store a drug solution in a predetermined storing space and where a micro nozzle ejecting the drug solution as a microjet to an outside is formed at one side thereof;
an elastic membrane which is disposed between the pressure chamber and the micro drug chamber to partition the pressure chamber and the micro drug chamber; and
a laser unit which irradiates the pressure driving liquid stored in the pressure chamber with laser to generate bubble in the pressure driving liquid,
wherein the laser unit irradiates a laser beam having an oscillation wavelength range of 2.8 μm to 3.0 μm.

2. The microjet drug delivery system according to claim 1, wherein the laser unit is an Er:YAG laser oscillation apparatus irradiating a laser beam having a wavelength of 2.94 μm.

3. The microjet drug delivery system according to claim 2, wherein the laser unit irradiates a laser beam having a wavelength of 2.94 μm and a pulse duration of 150 to 500 μs.

4. The microjet drug delivery system according to claim 3, wherein the laser unit irradiates a laser beam having a wavelength of 2.94 μm and a pulse duration of 200 to 300 μs.

5. The microjet drug delivery system according to claim 1, wherein the micro nozzle of the drug chamber has a diameter of 80 to 120 μm.

6. The microjet drug delivery system according to claim 1, wherein another substance is further dissolved or mixed into water used as a pressure driving liquid of the pressure chamber.

7. The microjet drug delivery system according to claim 6, wherein the pressure driving liquid is an aqueous electrolyte solution where an electrolyte is dissolved.

8. The microjet drug delivery system according to claim 7, wherein the electrolyte is a salt.
